(19) United States
(12) Patent Application Publication
(10) Pub. No.: US 2013/0041042 A1
(43) Pub. Date: Feb. 14, 2013

(54) POLYMERIC COMPOSITIONS AND ARTICLES OF MANUFACTURE FOR WOUND AND BURN TREATMENT AND OTHER USES

(71) Applicant: John S. Boyden, JR., Salt Lake City, UT (US)

(72) Inventor: John S. Boyden, JR., Salt Lake City, UT (US)

(21) Appl. No.: 13/204,936

(22) Filed: Aug. 8, 2011

(51) Int. Cl. A61K 47/30; C09J 5/02; A61L 15/26

(52) U.S. Cl. 514/772.3; 523/105; 522/74; 156/307.3

(57) ABSTRACT

Polymeric compositions for enhanced wound or burn treatment characteristics and other medical, industrial, recreational and domestic applications can be formulated as biocompatible, elastomeric, polymers. Such compositions can be used as advanced wound or burn care dressings or for other medical and non-medical purposes. The compositions generally comprise a polymeric component including a polyurethane oligomer or polymer with one or more hydroxyl-containing acrylate or methacrylate terminal groups thereon, and an effective amount of a mercaptan (thiol) to regulate elastic and adhesive properties of the cured composition. During medical uses, the composition may be applied in a thin layer on the affected area and a generous portion of the adjacent healthy skin. The composition is then cured such as by exposure to an external energy source, for example a visible light or an ultraviolet light or infrared light or sunlight or a combination thereof, to form an elastomeric dressing. When the healing process is completed, the dressing can then be peeled off the skin. Articles of manufacture incorporating the polymeric compositions, which can be used in various products, are also provided.

### BACKGROUND OF THE INVENTION

1. The Field of the Invention

This invention relates to polymeric compositions that can be cured to form a film suitable for a wound or burn dressing and for other medical, industrial, recreational, domestic and other applications. The invention also relates to articles containing these compositions and the process by which the compositions are made.

2. The Related Technology

Curable polymeric compositions are known to be useful for wound and burn treatment and related uses. U.S. Pat. Nos. 5,030,665 and 5,135,964 disclose a radiation-cured film that is useful as a surgical drape or wound dressing. The film includes a copolymer of a vinyl monomer and an acrylate capped polyurethane prepolymer. While the films disclosed in the these patents and related prior art, provide an alternative to evaporating a solvent to form a film, such films may not provide a combination of enhanced properties such as (a) the desired softness and elasticity necessary for comfort and freedom of movement while covering a wound or burn; (b) regulation of moisture vapor transmission rate (such as formulating for maximum, intermediate or minimum moisture transmission); (c) minimization or elimination of pain in first and second degree burns without the use of analgesic medication; (d) scab-minimized or scab-free healing; (e) scar minimization; (f) methods for enhancing adhesion to the skin; (g) minimization or elimination of cytotoxicity of both the uncured and cured versions of the compositions in cell cultures; and (h) the ability to cure thick layers of compositions provided by the preferred method using visible light.

Accordingly, there is a need for improved materials for use in wound and burn care products that overcome some or all of the above mentioned deficiencies of the prior art.

### SUMMARY OF THE INVENTION

The present invention is directed to polymeric compositions that provide enhanced wound or burn treatment and other characteristics useful in medical, industrial, recreational and domestic applications. The flexibility, elasticity, adhesion and adjustable moisture vapor transmission rate of these compositions and their methods of application meet multiple needs. The compositions can be formulated as biocompatible, elastomeric polymers that can be used by health care professionals, emergency responders and other persons as advanced wound or burn care dressings or for other purposes, such as anchoring medical devices, delivering drugs to the skin for local therapeutic purposes or for transdermal systemic drug absorption, for prevention of sores and enabling a more efficient and less-painful method for performing certain kinds of debridement.

The compositions of the invention provide an improvement in treatment of patients with wounds and or burns because the compositions can be applied in viscous form to the body and cured (as defined below) into a film that significantly reduces the pain and scarring typically associated with wounds and burns without the use of analgesic medication. The cured film also allows for a wide range of motion of joints because of the elasticity and firm adhesion of the film, enabling freedom of movement during the healing process. Different compositions of the invention have different moisture vapor transmission rates through the cured film when they are applied and cured. By selecting an inventive composition having the appropriate moisture vapor transmission rate, either wound desiccation or maceration can be treated or prevented. An inventive composition having a low moisture vapor transmission rate can be useful for softening pre-existing scabs and desiccated tissue prior to debridement. The compositions are naturally transparent, but they also can be made with varying degrees of translucency by using appropriate additives such as pigments or fine particles. The cured film also protects the wound or burn from contamination by foreign matter, decreases the risk of added trauma to the wound by its elasticity and softness, and enhances healing. The compositions of the invention can also be formed into precured articles such as transparent medical dressings. These articles are very flexible even at thicknesses that exceed the thickness of many existing transparent medical dressings. Thus, the inventive, precured dressing does not tend to cling to things during application in the manner exhibited by many transparent polymeric dressings so special packaging and expert application technique are not required to apply this product. Other uses of the compositions include powder-free gloves for both medical and non-medical uses, masks and parts thereof for use with continuous positive airway pressure devices, sunburn remedy, catheter tubing, shoes, shoe insoles, caulking and patching materials and various other adhesive applications.

The compositions of the invention generally comprise a polymeric component including a polyurethane polymer prepared by reacting a diisocyanate or blend of diisocyanates, a diol or blend of diols, a hydroxyacrylate or hydroxymethacrylate or a blend thereof and a capping agent(s) such as one or more of methoxypolyethyleneglycol(s) (MPEGs), a diol such as PEG 400 or a blend of MPEGs and diols such as PEGs. The compositions can be formulated for a high, intermediate or low moisture vapor transmission rate by varying the average molecular weight of the diol(s) and/or the hydrophilicity of the diol(s). After the polymer has been substantially reacted, a mercaptan is added to the compositions to regulate elastic and adhesive properties of the cured compositions by varying the concentration of mercaptan. Additional ingredients can be added to the compositions depending on the characteristics desired for the cured film.

In one embodiment of the invention, a composition, in viscous form, is applied as a layer on the surface of affected skin and adjacent healthy skin or on an area of healthy skin in need of protection. The composition's viscosity enables it to spread easily and conform to the curvature and to the minute details of the surface of the skin. The composition then can be cured, by exposure to actinic radiation, including but not limited to visible light or ultraviolet light or infrared light or sunlight (when appropriate photoinitiators are added to the composition), or by laser, chemical action, ultrasound, electron beam, electric current, ionizing radiation or heat, as commonly known in the art, to form an adherent, elastomeric film that conforms to the shape of the surface to which it has been applied. After serving its purpose, the film can be peeled off the skin.

Another aspect of the invention relates to articles of manufacture incorporating the inventive compositions. Such articles can be used as various wound or burn treatment products, decubitus ulcer prevention pads, anchors for medical devices and other medical and non-medical uses. Various articles may optionally be used with a layer of adhesive, uncured inventive composition or other adhesive. When the uncured adhesive layer is used, it binds the article to its intended location and position of use. When an article has such a layer of adhesive, uncured inventive composition or other adhesive applied at the time of manufacture, a release layer is applied to protect that uncured layer. When the release layer is removed and the article is in place, the adhesive layer may be “cured” as defined below.

The invention includes the compositions in both uncured and cured form and in combinations of cured and uncured forms.

These and other features of the present invention will become more fully apparent from the following description and appended claims, or may be learned by the practice of the invention as set forth hereinafter.

### BRIEF DESCRIPTION OF THE DRAWINGS

In order to illustrate the manner in which the above recited and other advantages and features of the invention are obtained, a more particular description of the invention briefly described above will be rendered by reference to specific embodiments thereof that are illustrated in the appended drawings, it being understood that these drawings depict only a few typical embodiments of the invention and are not therefore to be considered limiting of its scope.

FIG. 1 is a schematic depiction that illustrates one method of the invention in which an uncured layer of a composition (10) of the invention is applied to the skin (12) of a patient to cover a wound area (14), with the composition cured (as defined below) by a light source (16);

FIG. 2 is a schematic depiction of a medical device (18) on the skin of a patient (26), with an uncured composition of the invention (24) applied thereon and then cured to secure the medical device;

FIG. 3 is a schematic depiction of a catheter (20) inserted into a blood vessel (22), with an uncured layer of a composition of the invention (24) applied over and around the catheter to secure its three-dimensional position and seal its entry point when cured;

FIG. 4 is a schematic depiction of an uncured composition of the invention (24) applied to the skin surrounding an ostomy (27) and cured to protect the skin and, if required, to also secure an ostomy appliance (28);

FIG. 5 is a schematic depiction of a preformed article useful in, for example, shoe insoles, according to one embodiment of the invention; a layer of precured composition (32) is covered with a layer of uncured composition (35) that is covered by a thin layer of cured composition (36). While the insoles are out of the shoes, the person using the shoes stands on the insoles in stockings or bare feet while a light is shined through the bottom of a transparent platform and the insoles that penetrates them and cures the uncured layer. The insoles are then inserted into their respective shoes. Thus, the person has insoles that are custom shaped to the person's feet. Alternatively, the insoles can be made using only the precured composition (32). This will provide comfort, but not a custom fit.

FIG. 6 is a schematic depiction showing a cross-section of double-sided tape or wider, double-sided film comprised of a precured film (42) having uncured layers of inventive composition (45) applied to both surfaces (43 & 44); both uncured layers may be covered with release film (46);

FIG. 7 is a schematic depiction showing a cross-section of a preformed shape (50) that includes a layer of uncured inventive composition (52) encased between a first film release layer (54) and a second, more-adherent film layer (56), which have been formed into a desired shape (not shown here). During use, the packaged preformed shape can be applied by pulling apart the film layers, thus stripping off the one, less-adherent film layer while leaving the uncured shape still attached to the more-adherent film layer. The uncured shape is then applied to the skin in a desired position and exposed to visible or ultraviolet or infrared light or sunlight through the remaining film layer to cure the underlying uncured shape. After curing, the remaining film layer is pulled away leaving the cured shape securely positioned on the skin.

FIG. 8 is a schematic depiction of a transparent medical dressing where a precured film dressing (62) is coated on one side with a layer of uncured inventive composition (64) that acts as an adhesive. A release layer (66) covers the uncured composition, according to another embodiment of the invention; the release layer is removed and the dressing is then applied; the uncured composition (64) is then optionally cured to enhance adhesion to the skin; when this embodiment is produced in long, narrow strips, it becomes an adhesive tape where the release layer is removed prior to application;

FIG. 9 is a schematic depiction of a precured film dressing (70) (see also FIG. 8) applied over an ulcerated wound (74) and adjacent healthy skin (72);

FIG. 10 is a schematic depiction of the formation of a protective glove from a composition of the invention by curing the composition (80) already applied to the hands and cured by an external light source (82);

FIG. 11 is a schematic depiction of a precured film (91) applied over a chronic wound (92) that has extended into the subcutaneous tissue (96). The precured film (91) also covers a generous portion of surrounding, healthy skin (93). This precured film (91) has a layer of adhesive (94) uncured inventive composition that was applied to one side of the film (91) at the time of manufacture (See also FIGS. 8 & 9). Both the precured film (91) and the uncured adhesive (94) have high moisture vapor transmission rates. After this film (91) has been applied and the uncured adhesive (94) has been optionally cured, an additional layer (95), having a lower moisture vapor transmission rate, is applied on top of the precured film (91), but the additional layer (95) covers only the area bounded by the size, shape and location of the chronic wound (92) that is being treated. It does not cover the adjacent healthy skin (93). This additional layer (95) can be applied as uncured composition from a tube or other container or in the form of a second, precured film (See also FIG. 8) cut to match the area of the chronic wound. In either case, after the additional layer (95) has been applied, it is then cured. This additional layer (95), with its lower moisture vapor transmission rate, enables a moister, favorable environment for a healing chronic wound (92) while the first layer (91) maintains a drier, more favorable environment for the surrounding healthy skin (93).

### DETAILED DESCRIPTION

### Definitions

As used throughout this document:

“Polymer” as used herein means a large molecule (macromolecule) composed of repeating structural units typically connected by covalent chemical bonds and also includes a relatively lower molecular weight molecule with repeating structural units commonly referred to as an “oligomer” or “prepolymer” both of which terms are commonly known in the related art.
“Cure” or “curable” or “cured” or “curing” refers to the process of cross-linking between polymer chains and/or reacting terminal groups on polymer chains by actinic radiation, including but not limited to visible light or ultraviolet light or infrared light or sunlight (when appropriate photoinitiator(s) is/are added to the composition), or by laser, chemical action, ultrasound, electron beam, electric current, ionizing radiation or heat, as commonly known in the art.
“Precured” means all or part of an article of inventive composition that is cured into a specific shape before use. It may or may not be combined with additional, uncured inventive composition.
“Substantial” or “substantially” when used in reference to a quantity or amount of a material, or a characteristic thereof, refers to an amount that is sufficient to provide an effect that the material or characteristic was intended to provide. Similarly, “substantially” when used to refer to completion of a desired reaction means the total completion or the effective completion of the reaction so that the desired effect is achieved, and when used with respect to the absence or removal of a material in a composition, means that the material is either completely absent or removed from the composition or is present only in an amount small enough as to have no material effect on the composition.
Concentrations, amounts, and other numerical data may be presented herein in a range format. It is to be understood that such format is used merely for convenience and brevity and should be interpreted flexibly to include not only the numerical values explicitly recited, but also to include all the individual numerical values or sub-ranges encompassed within that range as if each numerical value and sub-range is explicitly recited. For example, a molecular weight range of about 200 to 1450 should be interpreted to include not only the recited numbers of 200 and 1450 but also to include individual molecular weights such as 300, 400, 800, 1000, and sub-ranges such as 200 to 400, 800 to 1000, etc.
“Mercaptan” means an organic molecule having at least one or more —S—H (sulfhydril) functional groups (also known as a “thiol”).
“PEG” means polyethylene glycol.
“PEGs” means polyethylene glycols having different average molecular weights, for example:
“PEG 1000” means polyethylene glycol average molecular weight about 1,000.
“PEG 600” means polyethylene glycol average molecular weight about 600.
“PEG 400” means polyethylene glycol average molecular weight about 400.
“PEG 300” means polyethylene glycol average molecular weight about 300.
“MPEG” means methoxypolyethylene glycol.
“MPEGs” means methoxypolyethylene glycols having different average molecular weights, for example:
“MPEG 550” means methoxypolyethylene glycol average molecular weight about 550.
“MPEG 350” means methoxypolyethylene glycol average molecular weight about 350.
“IPDI” means isophorone diisocyanate.
“IPTI” means isophorone triisocyanate.
“HEA” means 2-hydroxyethyl acrylate.
“HEMA” means 2-hydroxyethyl methacrylate
“TMPTMP” means trimethylolpropane tris(3-mercaptopropionate).
“PTMP” means pentaerythritol tetrakis(3-mercaptopropionate)
“DBTDL” means dibutyltin dilaurate.
“E4DMAB” means ethyl 4-(dimethylamino) benzoate F.W. about 193.24
“Sparge” means aerating the reaction mixture with a stream of clean, dry air providing (a) oxygen that has an inhibitory effect on free radicals that may form in the reaction mixture, (b) a modest cooling effect on reaction temperature, and (c) a small, positive air pressure inside the reactor to discourage airborne water and other contaminants from entering the reaction chamber through the chamber's open ports.
“SCFH” means standard cubic feet per hour.
“FTIR” means Fourier Transform Infrared Spectroscopy.

The present invention relates to specially designed polymeric compositions with enhanced treatment characteristics for burn treatment and wound care, for prevention of bedsores, blisters and cracked skin, for protective padding, and for anchoring medical devices to the skin. Other aspects of the invention relate to articles of manufacture incorporating the compositions and also the use of uncured compositions in various medical, industrial, recreational, domestic and other applications.

The compositions of the invention can be considered a platform technology because they can be formulated to meet a variety of specific needs. For example, the compositions can be used to form films with different adhesion, elasticity, moisture vapor transmission and anti-infective properties, which allow the composition to be employed in multiple protective and therapeutic ways.

The ingredients of the compositions can be prepared, mixed together and reacted in such a way that, in both uncured and cured states, they are minimally toxic or non-toxic in cell cultures. The uncured state is a viscous liquid having the desired consistency without the use of solvents. Room temperature viscosity can be increased or decreased, without using solvents, by increasing or decreasing the average molecular weight of the polymeric backbone chains. This is accomplished by changing the quantitative proportions among the diisocyanate(s) and backbone diol(s) and capping PEGs and/or MPEGs (if any) or by changing the molecular weights of the diol(s) or by doing both. The compositions described herein may be applied as a film that serves as a dressing on the surface of affected skin and adjacent healthy skin. Once a composition is cured, the transparent compositions permit easy monitoring of the healing process through the transparent elastomeric film without removing the dressing. When the dressing has served its purpose, the dressing can be peeled off the skin.

### Composition Formulations

The compositions of the invention are specially prepared urethane acrylates or urethane methacrylates designed to meet specific performance goals. They are prepared in a manner that is calculated to render the composition minimally toxic or non-toxic in cell cultures and to provide good shelf life for the uncured composition. The proportions of the ingredients described herein are calculated taking into consideration molar relationships and the number of reactive groups on each molecule of ingredient. The ingredients are chosen to achieve the desired viscosity of the uncured composition, the desired adhesion to the skin, the desired elasticity of the cured composition, the avoidance of toxicity, the degree of transparency or translucency of the cured composition and the desired moisture vapor transmission rate of the cured composition as will be understood by those of skill in the relevant art.

The compositions of the invention comprise the reaction product of one or more diisocyanates, one or more diols, herein designated “backbone diol(s),” a hydroxy acrylate and/or a hydroxy methacrylate to produce a prepolymer, and reacting the prepolymer with a capping compound comprising one or more methoxypolyethylene glycols (MPEGs), one or more diols or a blend of MPEGs and one or more diols. Preferably the reactions are carried out with the reactants in neat form, i.e. in the absence of solvent; however, the reactions can occur in the presence of a solvent. Suitable solvents are well known in the art of polyurethane chemistry.

Isophorone diisocyanate (IPDI) is the preferred diisocyanate. Many other diisocyanates known in the art may be substituted for IPDI, such as aromatic diisocyanates, aliphatic diisocyanates, alicyclic diisocyanates and combinations thereof including methylene diphenyl diisocyanate (MDI), toluene diisocyanate (TDI), hexamethylene diisocyanate (HDI) and the like. It is understood that the term “diisocyanate” may optionally include a small concentration of triisocyanate such as isophorone triisocyanate (IPTI) to provide a small portion of the isocyanato groups in the polymer backbone chains in order to vary the properties of the polymer. For example, IPTI can be added to increase tensile strength of the cured composition. Typically, triisocyanates may be added in an amount of up to about 10 wt % and preferably about 0.5-5 wt % by weight of the final composition, depending on the intended application of the composition.

The diisocyanate(s) and backbone diol(s) are preferably initially mixed and reacted in proportions to produce a “backbone” prepolymer in which there is an excess of isocyanato groups sufficient to ensure that the resulting prepolymer is substantially capped with free terminal isocyanato groups, typically at both ends.

Various diols such as polyglycols can be used as the backbone diol(s) to form the backbone prepolymer. Non-limiting examples of suitable diols include polyether glycols such as polyethylene glycol, polypropylene glycol (PPG), and polytetramethylene glycol (PTMEG), as well as polyester glycols such as polyethylene adipate and polycaprolactone, or mixtures thereof. Preferred backbone diols are PEGs having an average molecular weight of about 200 to about 1450, preferably about 300-1000 and blends thereof, such as a blend of PEG 600 and PEG 1000, preferably in a molar ratio of about 60 percent PEG 600 and about 40 percent PEG 1000. PEGs of higher molecular weight, e.g., 1000 to 1450 are typically blended with lower molecular weight PEGs, e.g., about 200 to 600. Thus, the backbone diol can be used in a mixture of average molecular weights depending upon the desired properties of the composition. For example, the backbone polymer chains can be formed using a PEG having a lower average molecular weight, such as about 200 to about 400 when lower moisture permeability (moisture vapor transmission rate) is desired for the composition. When a higher moisture vapor transmission rate is desired, the polymer chains can be formed using only PEGs having a higher average molecular weight, such as about 600 to about 1450. The moisture permeability (moisture vapor transmission rate) can be adjusted between the maximum and the minimum by using various combinations of PEG at different average molecular weights, such as from PEG at a lower average molecular weight and the remainder of the PEG at a higher average molecular weight. The moisture permeability (moisture vapor transmission rate) adjustment for the composition can be accomplished while still preserving the elastomeric properties of the cured composition. Use of a diol blend of differing molecular weights can have the beneficial effect of helping to prevent crystallization of the viscous compositions during storage before use. When PEG 1000 or higher molecular weight PEG is used exclusively, the risk of crystallization of the uncured composition exists. Other polyglycols such as polypropylene glycol and polytetramethylene glycol can be employed in preferred wider molecular weight ranges, e.g., about 250 to 4000 and 250 to 2900, respectively. In addition, it is understood that the term “backbone diol” as used herein should be interpreted to allow inclusion of a relatively minor amount of one or more triols as part of the backbone diol. Exemplary triols are glycerol, polycaprolactone triol and polypropylene glycol triol and similar triols known in the art. The triols may be used in amounts up to about 15 weight % and preferably about 1-7.5 weight % of the final composition. Typically, triols may be used to add tensile strength to the final composition in combination with or instead of the triisocyanate discussed above.

The hydoxyacrylate and/or hydroxymethacrylate is then added to react with the terminal isocyanato groups. The preferred hydroxyacrylate is HEA. Other hydroxyl-containing acrylates and/or methacrylates, and mixtures thereof are also suitable such as HEMA, hydroxypropyl and hydroxybutyl acrylates and methacrylates The hydroxyacrylate is added in a proportion to react with about 65 molar percent to about 80 molar percent of the terminal isocyanato groups, thus leaving about 20 molar percent to about 35 molar percent of the terminal isocyanato groups unreacted.

After confirming that the HEA has been substantially reacted, such as by FTIR, a capping hydroxyl-containing compound, such as an MPEG or a blend of MPEGs, is used to at least substantially cap the remaining, unreacted isocyanato groups. Preferably, to ensure capping within a reasonable time, the MPEG or blend of MPEGs is added in an excess of that needed to react with remaining unreacted isocyanato groups. As a representative example, a preferred blend of 50% MPEG 550 and 50% MPEG 350 (molar ratio) added in an amount such that there is an excess of each capping MPEG as illustrated below in Example 1 Table 1, e.g., preferably about 4.7 wt. %. Alternatively, a capping diol, such as a polyglycol, including polyether glycols or polyester glycols can be used to cap the unreacted isocyanato groups. Capping diols such as PEGs, polyethylene adipate and polycaprolactone may be used in amounts in excess of that necessary to cap the unreacted isocyanato groups. Preferred capping diols are PEGs. PEG, such as PEG 400 or PEG 300 or, for example, a 50%/50% molar ratio blend thereof, which may include, for example, a 4.7 wt % excess of each to at least substantially cap the remaining unreacted isocyanato groups after the HEA has been substantially reacted. When a single MPEG is used for capping, such as MPEG 550 or MPEG 350, the MPEG may include an excess of about, for example, 9.4 wt % to assure complete capping within a reasonable time. This is illustrated below in Example 2 Table 2.

After the above reaction steps are complete, a mercaptan is added to the finished reaction product. The preferred mercaptans are TMPTMP and/or PTMP but other mercaptans known in the art may also be used, for example, ethylene bis 3-mercaptopropionate, glycol dimercaptopropionate, octadecyl 3-mercaptopriopionate, methanethiol, ethanethiol, 1-propanethiol, 2-propanethiol, 2-methyl-2-propanethiol, 1-butanethiol, 2-butanethiol, 2-methyl-1-propanethiol, 1-pentanethiol, 1-hexanethiol, 1-heptanethiol, 1-octanethiol, 1-decanethiol, 1-dodecanethiol, 1-hexadecanethiol, 1-octadecanethiol, 1,2-ethanedithiol, 2,2-propanedithiol, and mixtures thereof. The mercaptan is added in an effective concentration to regulate the elasticity and adhesive properties of the cured compositions. The amount ranges from about 1 wt % to about 20 wt % and preferably from about 5 wt % to about 15 wt % of the final compositions. In general, the greater the amount of mercaptan added within this range, the greater the elasticity and adhesive properties of the composition. For example, use of a mercaptan in these amounts can yield cured compositions with an elongation at break of about 400% to about 1200%.

DBTDL is the preferred catalyst for the reaction between isocyanato groups and hydroxyl groups on the backbone diol(s), the capping MPEGs and diols and the hydroxyacrylates and/or hydroxymethacrylates. Other catalysts known in the art to catalyze this reaction may be used.

A major goal of the invention is to provide compositions, in both the uncured and cured states, in which toxicity in cell cultures is minimized or eliminated. Four or more of the ingredients (PEGs, MPEGs, IPDI, and HEA), can exert at least some adverse effects on the final compositions that contribute to cytotoxicity in cell cultures and affect the quality of the final compositions. The procedures described hereafter provide more detailed information on the preparation of the compositions of the invention and precautions to ameliorate toxic effects. Although in this description the reactants are primarily IPDI, selected PEGs, MPEGs and HEA, it is understood that other reactants as described herein and known in the art and varying proportions can be utilized in preparing the products of this invention without departing from the inventive concept. Moreover, less painstaking procedures can be followed to produce the inventive compositions that may be satisfactory for some of the uses mentioned herein, but the procedure described herein comprises a way to obtain optimal quality that will minimize or eliminate toxicity of the product in cell cultures.

PEGs at the average molecular weights mentioned herein can be pretreated before using them in the reaction with the urethane/acrylate. PEG 1000 is a solid at room temperature. PEG 600 is usually a viscous liquid at room temperature (freezing point range 18° C. to 23° C.). The primary adverse effect of PEGs and MPEGs on the compositions is their water content that may be specified by manufacturers as 0.5 wt % maximum. The quality of the uncured compositions of this invention is enhanced by removal of the water prior to beginning the reaction. As a precaution and as a further enhancement to the quality of the compositions, ionic residue (if any), which may be present, is also removed during the cleanup process. One method of removing ionic residue from PEG 1000 or PEG 600 is accomplished by dissolving it in an equal volume of distilled water and passing the solution through a three stage deionization apparatus containing respectively cationic resin, anionic resin, and mixed cationic/anionic resin. Thereafter, the deionized solution is passed through a wiped film still that removes water and any volatile compounds that may be present, thus producing extremely dry, pure, deionized PEG 1000 and PEG 600. As a result of these precautions, foam typically does not form in the reactor during the preparation of the inventive compositions and no anti-foaming agents are needed. Other, alternative methods for removing ionic residues and volatile residues, including water, are known in the art and optionally may be used.

PEG 400, PEG 300, MPEG 550 and MPEG 350 are liquid at room temperature and contain a small, but significant quantity of water that may be specified as 0.5 wt % maximum. In addition, they may contain ionic residues and volatile compounds. They can be distilled in a wiped film still without prior deionization. Ionic residues are left behind in the still. The vacuum will draw off volatile residue, including water, thus producing extremely dry, pure, deionized PEG 400, PEG 300, MPEG 550 and MPEG 350. Other, alternative methods for removing ionic residues and volatile residues, including water, are known in the art and optionally may be used.

During manufacture of the uncured compositions, free monomeric IPDI and HEA are incorporated into the compositions by creating conditions in the reaction that enable them to be fully or substantially reacted by providing excess quantities of their respective reaction partners. This may be accomplished by first adding all IPDI to the reactor at the beginning of the reaction together with the DBTDL catalyst in a single stage as described herein. A concentration of DBTDL catalyst of 0.10 wt % to 0.25 wt % of the total, calculated batch weight is sufficient.

The reaction temperature may be kept at the preferred temperature of approximately 20° C. by controlling the diol addition rate, by using a jacketed reactor where the circulating fluid is kept at 15° C. and by carefully aerating the mixture with dry, filtered room air having a dewpoint of −100° F. (−73° C.). The air filter may be about 0.01 micron. The airflow of the sparge is usually less than 50 SCFH at the beginning of the reaction. This low flow is intended to keep the sparge air outlet in the reactor open and free of IPDI monomer and to begin oxygenation of the reaction mixture to inhibit the formation of free radicals during the reaction. The temperature regulating methods mentioned herein apply to polymer batches weighing between 2,000 grams and 5,000 grams. As the batch weight increases beyond this range, alternative temperature control methods may need to be used as practiced in the art. The reaction is begun by slowly adding a blend of diols, e.g., PEG 1000 and PEG 600 to the IPDI. As the reaction composition thickens, the sparge is gradually increased to help maintain the about 20° C. reaction temperature until the prepolymer has been completed. Although the about 20° C. reaction temperature is used in an effort to optimize the quality of the resulting compositions, this temperature is not a limiting requirement of this invention, as higher temperatures up to the 40° C.-60° C. range may be employed.

Upon verification by FTIR that the diol or diol blend is substantially reacted, the process can proceed to the addition of the acrylate. At this stage of the reaction, the prepolymer backbone chains have been completed. These chains also possess terminal isocyanato groups, typically at both ends.

The hydroxyl-containing acrylate, e.g., HEA is then added at a moderate rate so as to avoid a sharp temperature spike in the reaction that might lead to premature gelation by provoking HEA's vinyl groups to react with one another. By raising the sparge to about 180 SCFH, and by raising the temperature of the jacket fluid to about 100° C., the reaction temperature reaches a maximum of about 70° C. At this point, the purpose of the sparge is two-fold: (1) It exerts a direct oxygen inhibition effect on radicals that may form in the reaction mixture, thus helping to prevent premature reactions among HEA's vinyl groups. (2) The positive air pressure inside the reactor discourages airborne water and other contaminants from entering the reaction chamber through its open ports. If HEA is added in an amount such that the number of its hydroxyl groups is equal to the number of terminal isocyanato groups, it is likely that some unreacted HEA will still be present after the reaction has proceeded for a reasonable period of time. Thus, preferably, the amount of HEA added is such that there are fewer HEA hydroxyl groups competing for a comparatively larger number of terminal isocyanato groups and the desired objective of substantially reacting HEA's hydroxyl groups within a reasonable time can be accomplished. The quantity of HEA can be lowered to as low as about 65% of the molar quantity that would be required to react with all terminal isocyanato groups while simultaneously retaining adequate tensile strength in the cured, final compositions. The preferred amount of HEA is 70% to 80% of the molar quantity that would be required to react with all terminal isocyanato groups. The cured final compositions made in this manner require less mercaptan to produce the desired elasticity and skin adhesion. Moreover, this lowered quantity of HEA yields cured final compositions that feel more like natural skin than compositions made with 100% of the molar quantity of HEA required to react with all terminal isocyanato groups. This method substantially improves the properties of the cured compositions discernible through touch. Again, FTIR is used to determine that the hydroxyl groups on HEA have been substantially reacted.

At this stage of the reaction, there is still a significant quantity of unreacted terminal isocyanato groups remaining. These excess, unreacted, terminal isocyanato groups can be capped by adding a quantity of MPEG or PEG or blends thereof sufficient to provide an excess of hydroxyl groups so as to obtain rapid and substantially complete capping. This may be accomplished by using a preferred blend comprised of, for example, a molar ratio of about 50% MPEG 550 to about 50% MPEG 350 or, alternatively, of capping diols in a molar ratio of 50% PEG 400 to 50% PEG 300. This mixture of MPEG 550 and MPEG350 or PEG 400 and PEG 300 also contributes to the variability of the length of those polymer chains that were not capped at both terminal groups by HEA, thus making a significant, additional contribution to prevention of crystallization of the uncured compositions during long-term storage. When using two MPEGs for capping, each MPEG may be added in an amount that includes an excess, for example, of between about 4 wt % and 5 wt % greater than their respective calculated molar quantities needed to match the remaining, unreacted isocyanato groups. This is illustrated in Example 1 Table 1. When a single MPEG is used for capping, such as MPEG 550 or MPEG 350, that MPEG may include an excess up to about 9.4 wt % to assure substantially complete capping within a reasonable time. This is illustrated below in Example 2 Table 2.

After the foregoing reaction has been completed and FTIR has confirmed that there are substantially no detectable isocyanato groups remaining, a mercaptan, e.g., (TMPTMP or PTMP) is added to markedly enhance elasticity and skin-adhesion of the cured composition. The TMPTMP or PTMP can be added in a concentration from about 1 wt % to about 20 wt %, and preferably from about 5 wt % to about 15 wt % of the final composition.

Photoinitiators can be added to the final compositions to promote light curing as defined above. Visible light is preferred to cure the composition because of its capacity to penetrate into very thick layers of uncured composition as compared to ultra-violet light's more limited capacity to penetrate. Suitable visible light photoinitiators include camphorquinone; phenylpropanedione; 2,4,6-trimethylbenzoyldiphenylphosphine oxide; and the like. When ultra-violet (UV) light is used to cure the composition, conventional UV photoinitiators may be employed, for example, IRGACURE® 184, (1-hydroxy-cyclohexyl-phenyl-ketone) is preferred. Other UV photoinitiators, such as 2,4,6-trimethylbenzoyldiphenylphosphine oxide, can be used with the composition of the invention and are known in the art. Moreover, a photoinitiator can be used that contains a hydroxyl group, thus enabling it to react with a portion of the terminal isocyanato groups on a polymer backbone chain. An example of this kind of “reactable” photoinitiator is IRGACURE 2959® (2-hydroxy-1-[4-(2-hydroxyethoxy) phenyl]-2-methyl-1-propanone).

The quantity of photoinitiator to be added to the compositions ranges from about 0.005 wt % to about 10 wt %, and preferably from about 0.01 wt % to about 4 wt % of the compositions.

Additional, optional ingredients may be added to the compositions to accelerate curing by visible light or UV light, such as ethyl 4-(dimethylamino) benzoate (E4DMAB). The E4DMAB can be used in an amount such as 0.5 wt % when the compositions are cured by visible light or UV light.

Alternatively, other curing agents may be added to the compositions of the invention to promote curing of the urethane/acrylate polymer chains in place of the photoinitiator when use of a light source for curing is not feasible or desirable. For example, a chemical initiator may also be used such as benzoyl peroxide. The chemical initiator is kept separate from the polymer compositions until curing is desired, at which time the chemical initiator and, optionally, an appropriate accelerator are then mixed with the polymer compositions using methods that are known to those skilled in the art. The quantity of such alternative initiators when added to the compositions are typically from about 0.3 wt % to about 10 wt % of the compositions, and preferably from about 2 wt % to about 6 wt %.

Other ingredients may be added to the compositions as desired. For example, an antioxidant can be added to help prevent the compositions from gelling prematurely by inhibiting free radicals. Suitable antioxidants include Irganox® 1035 from Ciba Geigy. Other common antioxidants are vitamin C (ascorbic acid) and vitamin E (tocopherols), and blends of antioxidants. The quantity of Irganox 1035 to be added to the compositions typically ranges from about 0.9 wt % to about 4 wt % and preferably from about 1.5 wt % to about 2.5 wt %. Others may be added in concentrations that reflect their relative activity as antioxidants.

The compositions of the invention can be formulated, as described herein, to exhibit a combination of some or all of the following characteristics: visual clarity of the uncured and cured compositions or translucency as desired (the compositions are naturally transparent, but they also can be made with varying degrees of translucency by using appropriate additives, for example, pigments or fine particles); water washability of the uncured polymer; very low degree of heat emanation during the curing process (to avoid discomfort and/or burns); firm adhesion to the skin; high elasticity of the cured material to enhance adhesion to the skin and to allow freedom of movement for the patient; soft hand or touch; compatibility with tackifying resins if desired; minimal or no irritation to the skin; different moisture vapor transmission rates of the cured films; and repellency to fluids and other sources of contamination; reduction or elimination of pain from first and second degree burns; enhanced wound healing including reduction of scarring. Additionally, the compositions of the invention can be sterilized by heat. The cured compositions allow self-sealing after drawing off excess fluid from a wound or burn with a small diameter hypodermic needle.

### Methods of Use of the Compositions

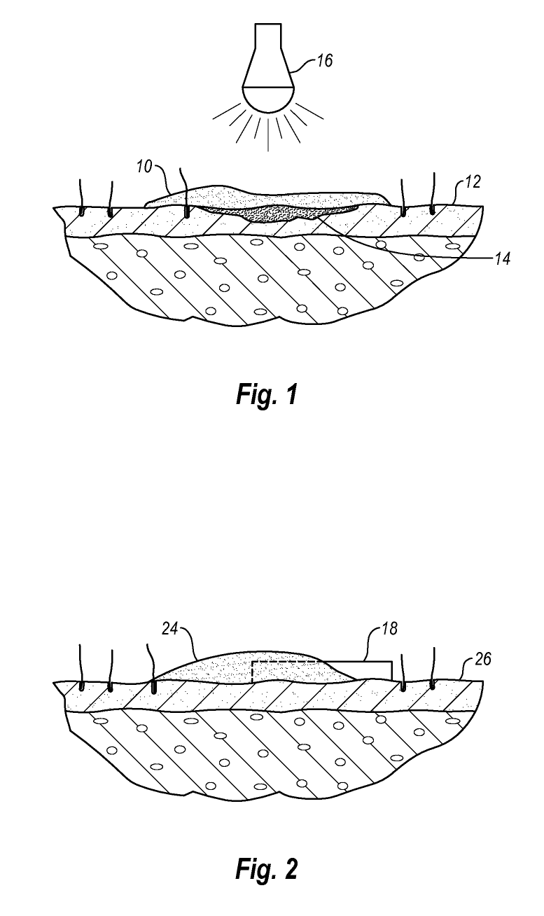

When using the curable polymer compositions of the invention as a wound dressing, burn treatment or surgical drape, the uncured compositions are applied as a film layer over the wound or burn area or surgical incision site and the adjacent healthy skin, such as shown in FIG. 1, where a layer of composition (10) applied to the skin (12) covers a wound or burn area (14) and a generous portion of adjacent healthy skin. The thin layer of the composition may be adjusted to any desired thickness by conventional methods, such as by use of a sterile wooden tongue depressor, and may have a thickness from about 0.1 mm to about 1.5 cm, and preferably from about 2 mm to about 7 mm. Where it is used as a padding to prevent bedsores or discomfort, it can be as thick as about 5 cm. if desired. The viscous composition spreads easily and conforms exactly to the minute details of the surface of the skin. The composition will readily stretch and bend with the skin to which it adheres.

When the uncured compositions have been applied, they can then be exposed to an energy source, such as a light source (16) in FIG. 1, for curing by visible light (e.g., about 395 nm to about 515 nm) preferably 430 nm to 485 nm, when the composition includes camphorquinone as a visible light photoinitiator. For example, it is preferred to use a light source such as a flashlight or lamp comprised of one or more light emitting diodes that emit light in the visible spectrum that corresponds to the wavelengths that are optimally absorbed by the photoinitiator, but a xenon lamp (optionally filtered for light output that falls within the wavelengths that are optimally absorbed by the photoinitiator), or a low powered laser that emits light within the aforementioned wavelengths or an incandescent lamp, also can be used. The compositions are typically cured by exposure to the energy source for about 3 to about 120 seconds, thereby forming very elastic, transparent films that adhere tightly to normal skin. Alternatively, a source of UV light can be used (e.g., long wave UV light at about 300 nm to about 395 nm) when the composition includes a UV photoinitiator. A conventional UV lamp may be used, as for example, a B-100 series Blak Ray® lamp from UVP, LLC of Upland, Calif. Sunlight also will initiate curing of the compositions whether the composition contains a visible light photoinitiator, a UV photoinitiator, an infrared photoinitiator or combinations thereof.

The length of exposure required to cure the liquid film compositions depends on the thickness of the film, the concentration of the photoinitiator, the energy of the light source, the distance of the light source from the composition, the percentage of light output within the absorbance spectrum of the initiator, the temperature of the composition, and the concentration of terminal vinyl groups. In general, films of from about 1 mm to about 1 cm in thickness are cured in about 5 to about 45 seconds when the lamp is positioned at a distance of about 10 inches from the film. Parameters of the curing such as lamp intensity, duration of exposure, distance of the film from the lamp, etc. are understood by those skilled in the art. In a formulation having a high moisture vapor transmission rate, such as a blend of PEG 1000 and PEG 600 diols, for example, the cured composition allows water vapor to pass therethrough while not allowing fluids to flow freely through it or bacteria to pass. When the polymeric chains of compositions are formulated with progressively shorter-length diols such as PEG 600, PEG 400 and PEG 300, for example, the cured compositions have progressively lower moisture vapor transmission rates.

Once the compositions are cured, easy monitoring of the condition of the skin or wound or burn during the healing process can be done by observing the skin or wound or burn through the transparent film without removal thereof. Even after many days of use of the film having the high moisture vapor transmission rate, the condition of the skin remains good under the cured film with minimal or no skin softening, wrinkling and loss of color that are associated with the use of adhesive tape under similar circumstances. When an occlusive dressing version of the composition is cured, significant moisture will accumulate under the dressing. This version of the composition is not intended for routine long-term use. It is useful for softening pre-existing scabs and desiccated tissue prior to debridement and for administering topical or transdermal medication for a brief period of time. When the healing process or other purpose has been completed, the film dressing can be peeled off the skin.

The compositions of the invention can be applied directly to the skin of a patient in several forms, such as in a spray or a viscous liquid. The compositions may be supplied in a squeezable tube and squeezed from the tube onto the skin; applied from a wide-mouthed jar or other container using a sterile tongue depressor or other instrument; or from a shaving cream-type pressure can; or by a device designed especially to apply the compositions. The viscosity of the composition can be altered to suit the intended use and the method of application.

The compositions of the invention provide various benefits and advantages to the caregiver or user. The compositions are readily applied. In their usual embodiments, no mixing of ingredients is required at the time of application and the compositions have a rapid curing time. If the user needs unanticipated extra time to adjust a device after the composition has been applied in its viscous liquid state, the user may cure the composition when the user is ready. In addition, toxic, noxious or volatile solvents that could have an adverse effect on the patient, the immediate environment, or the person applying the compositions are not present in the compositions.

The compositions of the invention cured into a film can be used as adherent dressings for surgical wounds, cuts, abrasions, decubitus ulcers, diabetic ulcers, and burns. They can also be used as a protective dressing for normal skin to help prevent blisters, dermal calluses, cuts, abrasions, and decubitus ulcers.

The compositions are particularly effective when used as dressings for burns. The pain of second-degree burns is usually markedly reduced or eliminated by application and curing of the compositions directly on the burn site. If treated early enough, the burn may heal without scabs. Scarring is typically markedly reduced from what otherwise would be expected. Dressing changes are minimized. If the burn has scabs at the time of initial treatment, a low moisture vapor transmission rate composition can be used as the dressing to minimize or eliminate the pain and to retain moisture so that the scabs can soften and partially liquefy into a gelatinous form that is more easily removed. Upon removal, a new dressing using a high moisture vapor transmission rate composition can be applied.

The cured film of the compositions can be used to hold skin edges together on many lacerations and surgical wounds. It can be initially applied by using either a viscous liquid composition or a precured shape (later described herein). In all cases, the composition or precured shape adheres to the surface of the skin and is not used within the wound as surgical glue (tissue adhesive). In addition, the film can be used in combination with absorbable or non-absorbable sutures or staples or other wound closure devices. The compositions covering the wound enable scabless healing with its accompanying decreased scarring and accelerated healing time. Because the cured film keeps external fluids out of the wound, the patient is able to shower much sooner after surgery than he or she could if gauze were used as the wound dressing. All of the other advantages of the compositions mentioned elsewhere herein apply to surgical wounds, such as the ability to monitor wound healing by visualization through the transparent dressing and improved post-operative mobility. The flexibility in applying the compositions of the invention provides a secure, effective dressing even under difficult circumstances where the need for drainage or other special considerations arises.

In another aspect of the invention, the compositions can be applied to the body of a patient to provide an adherent protective coating for securing a medical device in place on the patient's body. For example, the compositions can be very useful adhesives for anchoring and protecting diagnostic or treatment devices on the skin, such as needles, intravascular and urinary catheters, arterio-venous fistulae, ostomy bags, electrodes, metering devices, mediastinal tubes, pleural cavity tubes, tubing for metered insulin administration, and other medical devices. FIG. 2 represents a medical device (18) on the skin (26) of the body of a patient, with a composition (24) applied thereover so as to cover at least a portion of the medical device (18). The composition (24) is cured as discussed above to form an adherent protective film that secures a medical device (18) in place on the body of the patient.

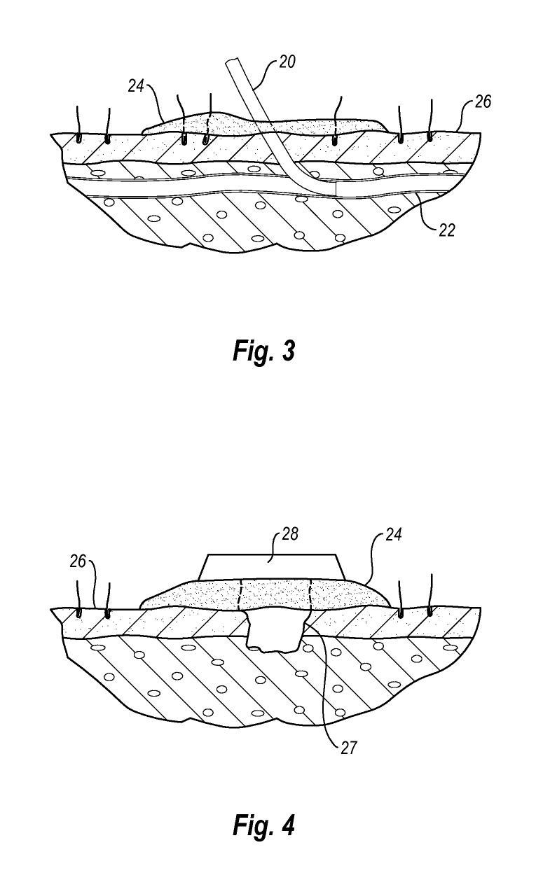

When used as a vascular catheter anchor to the skin, the composition should have a high moisture vapor transmission rate. After the catheter has been inserted, the composition is applied directly on the puncture site and surrounding healthy skin. While the catheter is held at the desired angle in relation to the skin, the light is shined on the composition to cure it into its solid state, thus stabilizing the catheter in the desired position. A schematic depiction of such a configuration is shown in FIG. 3, where a catheter (20) has been inserted into a blood vessel 22 in the body of a patient. A composition (24) is applied and is cured over the puncture site where the catheter (20) enters the skin (26). By steadying the catheter in the desired position and at the desired angle to the skin during the curing process, the position and angle of the catheter are secured. Because the composition has the capacity to absorb and transmit moisture to the air in significant quantities, the skin adjacent to the puncture site is kept humid, but ordinarily not wet. Further, the cured composition prevents the catheter from sliding in and out of the puncture site, removing a possible mechanism of contamination and/or trauma to the inner wall of the blood vessel. These same principles apply to a variety of catheter locations including central venous pressure lines and subclavian vein catheters. By further use of the composition, catheter tubing may be secured to the patient and to other nearby locations, such as a bed rail, as desired to provide additional protection and stability.

When used as ostomy appliance adhesives, the compositions are applied in a thick layer to the skin surrounding the os and for a radius that exceeds the radius of the base of the ostomy appliance (such as a collection bag). The composition can be further applied to any surrounding skin that is irritated or otherwise needs a protective cover. Upon curing with the light source, the bag is held securely and without leaks even in areas where the skin surface is irregular. Such a configuration is shown in FIG. 4, where a composition (24) is applied to the skin (26) surrounding the ostomy (27) and for a radius that exceeds the radius of an ostomy appliance (28). Additionally, the compositions can be used as protective coatings for skin surrounding fistulae or other openings from which irritating and/or infective substances flow.

The compositions of the invention can further be used as surgical wound dressings. The compositions are applied as wound dressings to prevent or reduce scarring and to provide a barrier to contamination after surgical procedures. In some cases, the compositions can also be used as emergency room suture substitutes. The compositions can be used to close minor lacerations that would otherwise require skin sutures, tape or surgical glue (tissue adhesive). When used as a suture substitute, the composition eliminates the need for local anesthesia. The inventive compositions are used on the surface of the skin and not in the wound. They are not surgical glue (tissue adhesives).

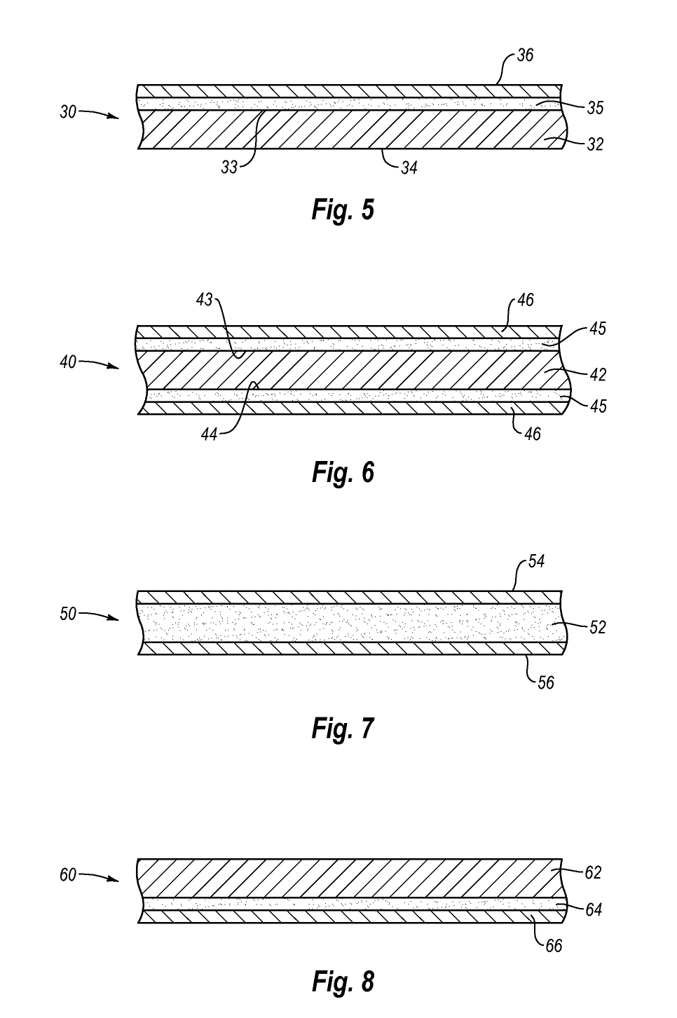

The articles in FIGS. 5, 6, 7, 8, 9 & 11 presuppose opaque overwraps (not shown) to protect them from accidental exposure to light that would result in curing before the end users are ready to use them. Precured compositions according to the invention can also be used together with adhesive coatings of uncured composition on preformed articles such as single or double-sided tape, bandages, shoe insoles or other reinforcing structures as shown in FIGS. 5 & 6.

The article in FIG. 7 shows a cross-section of a preformed shape (50) that includes a layer of uncured inventive composition (52) encased between a first film release layer (54) and a second, more-adherent layer (56), which have been formed into a desired shape (not shown here). During use, the packaged preformed shapes can be opened by removing the opaque overwrap and pulling apart the film layers, thus stripping off the one, less-adherent film layer while leaving the uncured shape still attached to the other film layer. The uncured shape is then applied to the skin in a desired position and exposed to visible or ultraviolet or infrared light or sunlight through the remaining film layer to cure the underlying uncured shape. After curing, the remaining film layer is pulled away leaving the cured shape securely positioned and attached to the skin. This article is useful for administering local or systemic medication.

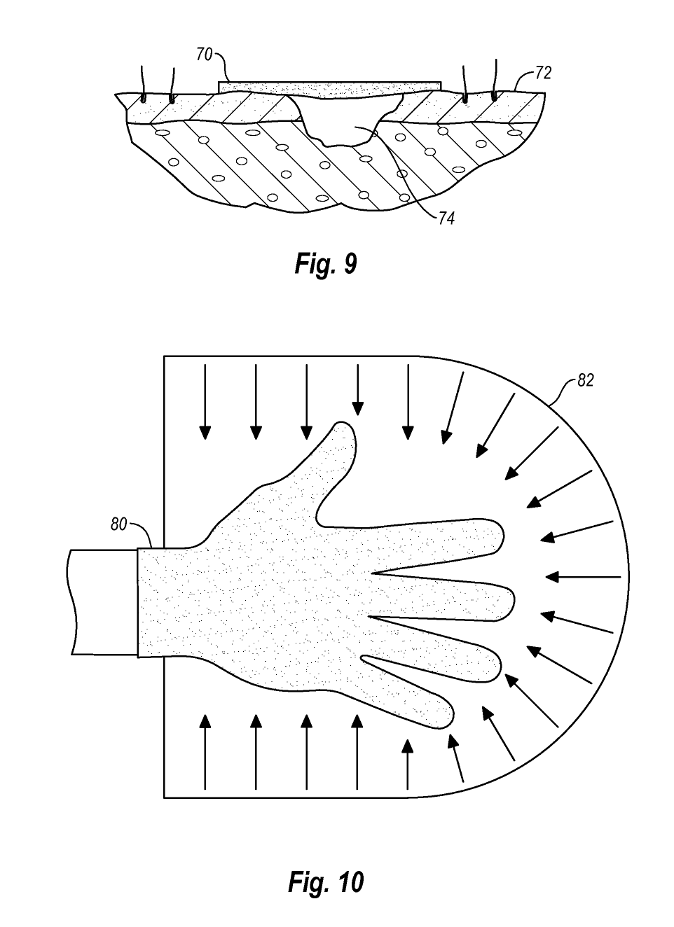

The article in FIG. 8 is a precured film made of an inventive composition (62), coated on one side by an adhesive layer of uncured inventive composition (64) that is covered by a release layer (66). The precured film dressing can be applied by removing the release layer and placing the precured film substrate, adhesive side down, on the desired location on the skin. If desired, a visible light or an ultraviolet light then optionally may be shined through the precured film to cure the adhesive layer, enhancing its adherence to the skin. Such precured shapes are particularly useful as transparent medical dressings for cuts, abrasions, small burns and also in ulcerated wounds where the dressing needs to stretch across the rim of the ulcer without filling in the concavity thereof so that the natural healing process will be encouraged to progress under the dressing. For example, FIG. 9 shows a precured dressing (70) (also see FIG. 8) applied onto skin (72) over an ulcerated wound (74). The precured dressing (70) covers the wound (74) without filling in the concavity of the wound.

The compositions may also be used to prepare preformed articles such as gloves. For example, persons can dip their hands into a vessel containing the composition in its liquid form or spread the uncured composition on their hands after obtaining a suitable amount of the liquid from a dispenser, and then place their hands in a position for curing the composition by an external source of energy. FIG. 10 depicts the formation of a protective glove (80) from a composition of the invention by use of a light source (82), such as a light-producing tunnel or hood that exposes the uncured glove to light at all angles simultaneously for curing. Moreover, an alternative method for producing protective gloves is to spray uncured composition onto forms that are shaped like human hands and to immediately cure them by one of the methods described above in Definitions.

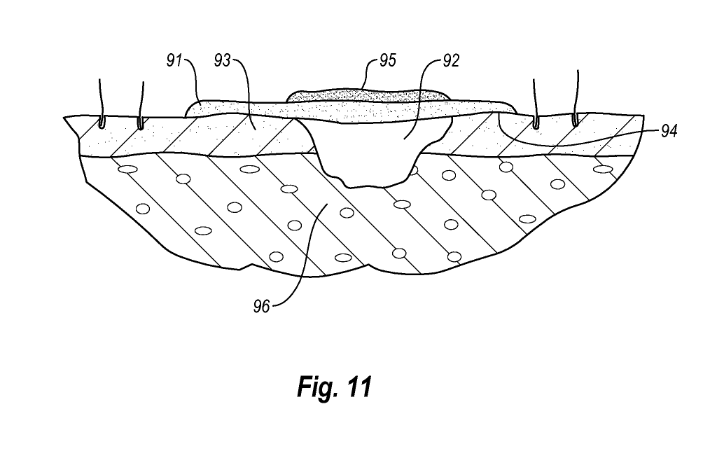

An important embodiment of this invention combines the wound protection/healing promotion properties of the compositions with the additional benefit of keeping the adjacent healthy skin relatively dry. As stated earlier in this application, FIG. 11 is a schematic depiction of a cross section of a precured film (91) (also see FIGS. 8 & 9) applied over a chronic wound (92) that has extended into the subcutaneous tissue (96). The precured film (91) also covers a generous portion of surrounding, healthy skin (93). This precured film (91) has a layer of adhesive (94) uncured inventive composition that was applied to one side at the time of manufacture. Both the precured film (91) and the uncured adhesive (94) have high moisture vapor transmission rates and are thus substantially permeable. After this film (91) has been applied, an additional layer (95), having a lower moisture vapor transmission rate and thus substantially less permeable, is applied on top of the precured film (91), but the additional layer (95) covers only the area bounded by the size, shape and location of the chronic wound (92) that is being treated. It does not cover the adjacent healthy skin (93). This additional layer (95) can be applied either in the form of an uncured viscous inventive composition (and later cured) or in the form of a second, precured film cut to match the area of the chronic wound. This additional layer (95), with its lower moisture vapor transmission rate, enables a moister, favorable environment for a healing chronic wound (92) while the first layer (91) maintains a dryer, more favorable environment for the surrounding healthy skin (93).

### EXAMPLE 1

A polymeric composition curable with visible light can be formulated according to the methods of the invention with the ingredients and quantities listed in Table 1 below. This formula provides a cured film with a relatively high moisture vapor transmission rate and is suited for most wound dressing and burn applications. The process for preparing this composition is described below.

TABLE 1

| | Quantity | Wt % in |
|---|---|---|
| Ingredient | (grams) | batch |
| Diisocyanate (IPDI) | 669.41 | 22.31% |
| DBTDL | 2.10 | .07% |
| Irganox 1035 | 60.0 | 2.00% |
| PEG 1000 | 866.94 | 28.90% |
| PEG 600 (add simultaneously with | 780.25 | 26.01% |
| PEG 1000 as a blend) | | |
| Hydroxyethyl acrylate (HEA) | 146.99 | 4.90% |
| MPEG 550 (capping, including a 4.74 | 121.56 | 4.05% |
| wt % excess of MPEG 550) | | |
| MPEG 350 (capping, including a 4.74 | 77.36 | 2.58% |
| wt % excess of MPEG 350) (add | | |
| simultaneously with MPEG 550 as a blend) | | |
| DBTDL | 0.90 | .030% |
| TMPTMP | 270.00 | 9.00% |
| Camphorquinone | 4.50 | 0.15% |
| E4DMAB (optional) | 0 | 0% |
| TOTAL | 3,000,00 | 100% |

The following process is carried out in which the batch described in Table 1, above, weighing 3,000.00 grams is prepared. Initially, the diisocyanate is added and then aerated (sparged) with clean, dry air while gently mixing in the DBTDL catalyst and the antioxidant. The sparge air initially flows at less than 50 standard cubic feet per hour to avoid aerosolizing the liquid starting materials. The air comes from an oil-free compressor and is dried to a dewpoint of −100° F. and passed through a filter having a 0.01 micron pore size. This minimizes or prevents introduction of moisture and both biological and non-biological contaminants from the compressed air and provides a small, positive air pressure inside the reactor to discourage airborne water and other contaminants from entering the reaction chamber through its open ports.

The diol blend (PEG 1000 and PEG 600) is added to the diisocyanate/catalyst/antioxidant mixture slowly to help control the reaction temperature. The reaction temperature is controlled by keeping the fluid in the heating/cooling jacket of the reaction vessel at 15° C. and by keeping the drop-wise addition of the diol blend slow enough to avoid overheating by the exothermic reaction between the diol blend and the diisocyanate. The reaction temperature is not allowed to rise above 20° C. during the addition of the diol until FTIR analysis shows that reactive hydroxyls of the diol blend are substantially undetectable. The sparge airflow is then increased to 180 standard cubic feet per hour (SCFH) to aerate the reaction mixture for about 15 minutes before addition of hydroxyethyl acrylate (HEA), and the temperature of the fluid in the heating/cooling jacket is raised simultaneously to 100° C.

The temperature in the heating/cooling jacket is kept at 100° C. The reaction temperature is kept at or below 80° C. primarily by the rate of addition of HEA. When FTIR analysis shows that the hydroxyls on the HEA are substantially reacted, the blend of capping MPEGs (MPEG 550 and MPEG 350) is rapidly added. The second addition of catalyst begins after the capping MPEGs have been stirred into the reaction mixture for about 5 minutes. The temperature in the jacket is kept at 100° C. until the isocyanato groups are substantially capped as verified by FTIR spectrometry.

The Mercaptan (TMPTMP) is added to the composition after the capping is substantially completed and the jacket temperature is decreased to 80° C. At this point, measures should be taken to assure that light cannot enter the reaction vessel (for example, a glass reaction vessel can be completely covered with aluminum foil) before adding the photoinitiators. The preferred photoinitiator, camphorquinone, is then added and mixed for about 15 minutes. At this point, the viscous composition of the invention is complete. The sparge air is left on and the jacket temperature remain at 80° C. until the composition in the reaction chamber has been drained into opaque containers. They can be filled under conditions of faint, red light to avoid premature curing of the composition. Heat sterilization, if required, can then be carried out.

### EXAMPLE 2

A second polymeric composition curable with visible light is formulated according to the methods of the invention with the ingredients and quantities listed in Table 2 below. In contrast to Example 1, this formula provides a cured film with a relatively low moisture vapor transmission rate and is suited for occasions when an occlusive wound or burn dressing is desired. It is also useful in providing a temporary covering for wounds where debridement is to be performed. The occlusive nature of this film retains moisture that is escaping from the skin and softens skin and scabs. If left in place long enough, scabs will become very hydrated or even jelly-like, thus reducing the pain associated with debridement. The process for preparing this composition is described below.

TABLE 2

| | Quantity | Wt % in |
|---|---|---|
| Ingredient | (grams) | batch |
| Diisocyanate (IPDI) | 961.63 | 32.05% |
| DBTDL | 2.10 | .070% |
| Irganox 1035 | 60.0 | 2.0% |
| PEG 400 | 1387.97 | 46.27% |
| Hydroxyethyl acrylate (HEA) | 149.09 | 4.97% |
| MPEG 350 (capping, including a | 163.82 | 5.46% |
| 9.3 wt % excess of MPEG 350) | | |
| DBTDL | 0.9 | .03% |
| TMPTMP | 270.00 | 9.00% |
| Camphorquinone | 4.50 | 0.15% |
| E4DMAB (optional) | 0 | 0% |
| TOTAL | 3,000.00 | 100% |

The following process is carried out in which the batch described in Table 2, above, weighing 3,000.00 grams is prepared. Initially, the diisocyanate starting material is added and then aerated (sparged) with clean, dry air while gently mixing in the DBTDL catalyst and the antioxidant. The sparge air initially flows at less than 50 standard cubic feet per hour to avoid aerosolizing the liquid starting materials. The air is from an oil-free compressor and is dried to a dewpoint of minus 100° F. and passed through a filter having a 0.01 micron pore size. This minimizes or prevents introduction of moisture and both biological and non-biological contaminants from the compressed air and provides a small, positive air pressure inside the reactor to discourage airborne water and other contaminants from entering the reaction chamber through its open ports.

The diol (PEG 400) is added to the diisocyanate/catalyst/antioxidant mixture slowly to help control the reaction temperature. A low reaction temperature also is maintained by keeping the fluid in the heating/cooling jacket of the reaction vessel at 15° C. The reaction temperature is not allowed to rise above 20° C. during the addition of the diol until FTIR analysis shows that reactive hydroxyls of the diol (PEG 400) are substantially undetectable. The sparge airflow is then increased to 180 standard cubic feet per hour to aerate the reaction mixture for about 15 minutes before addition of hydroxyethyl acrylate (HEA), and the temperature of the fluid in the heating/cooling jacket is simultaneously raised to 100° C.

The reaction temperature is kept at or below 80° C. by adding HEA at a rate that enables that result. When FTIR analysis shows that the reactive hydroxyls on the HEA are substantially undetectable, the capping MPEG 350 is rapidly added. The second addition of catalyst begins after the capping MPEG 350 has been stirred into the reaction mixture for about 5 minutes. The temperature in the jacket is kept at 100° C.

The Mercaptan (TMPTMP) is added to the composition after the capping is substantially completed, as shown by FTIR, and the jacket temperature is decreased to 80° C. At this point, measures are taken to assure that light cannot enter the reaction vessel (in this case, the glass reaction vessel is completely covered with aluminum foil) before adding the photoinitiator. The photoinitiator is then added and mixed for about 15 minutes. At this point, the viscous composition of the invention is complete. The sparge air is left on and the jacket temperature remains at 80° C. until the composition in the reaction chamber has been drained into opaque containers. The containers are filled under conditions of faint, red light to avoid premature curing of the composition. Heat sterilization, if required, can then be carried out.

What is claimed is:

1. A polymeric composition comprising:
a) the reaction product of one or more diisocyanates, one or more backbone diols, one or more acrylates selected from the group consisting of hydroxy acrylates and hydroxy methacrylates to produce a prepolymer, said reaction product being further reacted with one or more hydroxyl-containing capping compounds selected from the group consisting of
MPEGs, polyglycols, and mixtures thereof, and
b) an effective amount of a mercaptan to regulate elastic and adhesive properties of the composition.

2. The composition of claim 1, wherein the one or more diisocyanates are selected from the group consisting of aromatic diisocyanates, aliphatic diisocyanates, alicyclic diisocyanates, and mixtures thereof.

3. The composition of claim 2, wherein the one or more diisocyanates further comprise one or more triisocyanates.

4. The composition of claim 2, wherein the one or more diisocyanates are selected from the group consisting of isophorone diisocyanate, methylene diphenyl diisocyanate, toluene diisocyanate, hexamethylene diisocyanate, and mixtures thereof.

5. The composition of claim 1, wherein the one or more backbone diols further comprise one or more triols.

6. The composition of claim 1, wherein the backbone diol is one or more poly ether glycols selected from the group consisting of polyethylene glycol, polypropylene glycol, polytetramethylene glycol, and mixtures thereof.

7. The composition of claim 6, wherein the one or more backbone diols comprise one or more polyethylene glycols having a molecular weight of about 200-1450.

8. The composition of claim 7, wherein the one or more diols comprise a first PEG with a lower average molecular weight of about 200 to about 600 and a second PEG with a higher average molecular weight of above about 600 to about 1450.

9. The composition of claim 8, wherein the polyethylene glycol is a blend of PEG 600 and PEG 1000.

10. The composition of claim 1, wherein the one or more acrylates are selected from the group consisting of hydroxyethyl acrylate, hydroxyethyl methacrylate, hydroxypropyl acrylate, hydroxypropyl methacrylate, hydroxybutyl acrylate, hydroxybutyl methacrylate, and mixtures thereof.

11. The composition of claim 1, wherein the mercaptan comprises a poly-thiol compound.

12. The composition of claim 1, wherein the mercaptan is selected from the group consisting of trimethylol propane tris(3-mercaptopropionate), pentaerythritol tetrakis(3-mercaptopropionate), and mixtures thereof.

13. The composition of claim 1, wherein the mercaptan is selected from the group consisting of ethylene bis(3-mercapatopriopionate), glycol dimercaptopropionate, 1,2-ethanedithiol, 2,2-propanedithiol, and mixtures thereof.

14. The composition of claim 1, wherein the mercaptan is selected from the group consisting of methanethiol, ethanethiol, 1-propanethiol, 2-propanethiol, 2-methyl-2-propanethiol, 1-butanethiol, 2-butanethiol, 2-methyl-1-propanethiol, 1-pentanethiol, 1-hexanethiol, 1-heptanethiol, 1-octanethiol, 1-decanethiol, 1-dodecanethiol, 1-hexadecanethiol, 1-octadecanethiol,, and mixtures thereof.

15. The composition of claim 1, wherein the mercaptan comprises about 1 wt % to about 20 wt % of the composition.

16. The composition of claim 1 wherein the hydroxyl capping compound is MPEG.

17. The composition of claim 1, wherein the one or more diisocyanates comprise IPDI, the one or more backbone diols comprise a PEG having an average molecular weight of about 200-1000 or a blend of PEGs within said range, the one or more acrylates comprise HEA, the hydroxyl containing capping compound is selected from the group consisting of a MPEG and a PEG and the mercaptan is TMPTMP, PTMP or mixtures thereof.

18. The composition of claim 17 wherein the PEG is a blend of PEG 1000 and PEG 600.

19. The composition of claim 18 wherein the one or more hydroxyl-containing compounds is a MPEG.

20. The composition of claim 1 wherein the reaction product of a) is obtained as follows:
i) an excess of the one or more diisocyanates is reacted with the one or more backbone diols;
ii) a compound selected from the group of a hydroxyacrylate, hydroxymethacrylate, and mixtures thereof is reacted with the product of step i) in proportions such that the resulting product contains free isocyanato groups;
iii) at least one diol or MPEG capping compound is added to the product of step ii) in proportions to substantially react with all free isocyanato groups;
and the mercaptan is added to the reaction product of step iii).

21. The composition of claim 20 wherein the one or more diisocyanates comprise IPDI, and the one or more diols in step i) comprise a PEG having an average molecular weight from about 200-1000.

22. The composition of claim 20 wherein the one or more diisocyanates comprise IPDI, the one or more diols in step i) comprise a blend of PEG 1000 and PEG 600, the hydroxyacrylate is HEA, and the mercaptan is TMPTMP, PTMP, or mixtures thereof.

23. A process for preparing an acrylate polyurethane comprising:
a) reacting a diisocyanate with a backbone diol, wherein the diisocyanate is provided in an amount such that the resulting reaction product is a prepolymer substantially capped with isocyanato groups;
b) reacting the product of step a) with at least one of a hydroxyacrylate or hydroxymethacrylate in proportions such that the resulting product is a prepolymer with free isocyanato groups;
c) reacting the product of step b) with at least one of MPEG, PEG or blends thereof in proportions such that there is an excess of hydroxyl groups in the resulting product; and
d) adding a mercaptan to the product of step c) in an amount of about 1 wt % to about 20 wt % of the final composition.

24. The process of claim 23, wherein the backbone diol of step a) is a blend of PEGs having a molecular weight of about 200-1000 and the mercaptan is PTMP, TMPTMP, or mixtures thereof.

25. The process of claim 23, wherein the reactant of step c) is MPEG.

26. A wound or burn care dressing comprising the polymeric composition of claim 20.

27. A method of securing a medical device to the body of a patient, comprising:
a) contacting the composition of claim 1 with at least a portion of a medical device; and
b) curing the composition to form an adherent protective film to secure the medical device on the body of a patient.

28. An article of manufacture comprising the composition of claim 1 formed into a predetermined shape.

29. An article according to claim 28 comprising a cured composition of claim 1 having coated thereon an uncured composition of claim 1.

30. A process for treating a wound or burn comprising:
applying a first layer of precured composition of claim 1 having a relatively higher moisture vapor transmission rate over both the wound and adjacent healthy tissue and applying an additional layer of said composition having a lower moisture vapor transmission rate over the area substantially bounded by the size, shape and location of the wound or burn being treated.

31. The cured composition of claim 1.

32. The cured composition of claim 17.

33. The cured composition of claim 19.

34. The process of claim 23 further comprising curing of the product of step d).

35. The process of claim 34 wherein the product of step d) is cured by exposure to visible light.
